(19) United States
(12) Patent Application Publication
(10) Pub. No.: US 2007/0110810 A1
(43) Pub. Date: May 17, 2007

(54) Transdermal drug delivery systems, devices, and methods employing hydrogels

(75) Inventor: Gregory A. Smith, Sammamish, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Transcutaneous Technologies Inc., Shibuya-ku (JP)

(21) Appl. No.: 11/541,389

(22) Filed: Sep. 27, 2006

Related U.S. Application Data
(60) Provisional application No. 60/722,789, filed on Sep. 30, 2005.

(51) Int. Cl. A61N 1/30; A61K 9/14

(52) U.S. Cl. 424/486; 424/487; 604/20

(57) ABSTRACT

Systems, devices, and methods for transdermal delivery of one or more therapeutic active agents to a biological interface. An iontophoretic drug delivery system is provided for transdermal delivery of one or more therapeutic active agents to a biological interface of a subject. The iontophoretic drug delivery system includes at least one active agent reservoir. In some embodiments, the at least one active agent reservoir include a backbone modified hydrogel matrix for incorporation one or more active agents.

### CROSS-REFERENCE TO RELATED APPLICATION

This application claims the benefit under 35 U.S.C. § 119(e) of U.S. Provisional Patent Application No. 60/722,789 filed Sep. 30, 2005, the contents of which are incorporated herein by reference in their entirety.

### BACKGROUND

1. Field

This disclosure generally relates to the field of iontophoresis and, more particularly, to transdermal drug delivery systems, devices, and methods employing hydrogel matrices.

2. Description of the Related Art

Iontophoresis employs an electromotive force and/or current to transfer an active agent (e.g., a charged substance, an ionized compound, an ionic a drug, a therapeutic, a bioactive-agent, and the like), to a biological interface (e.g., skin, mucus membrane, and the like), by applying an electrical potential to an electrode proximate an iontophoretic chamber containing a similarly charged active agent and/or its vehicle.

Iontophoresis devices typically include an active electrode assembly and a counter electrode assembly, each coupled to opposite poles or terminals of a power source, for example a chemical battery or an external power source. Each electrode assembly typically includes a respective electrode element to apply an electromotive force and/or current. Such electrode elements often comprise a sacrificial element or compound, for example silver or silver chloride. The active agent may be either cationic or anionic, and the power source may be configured to apply the appropriate voltage polarity based on the polarity of the active agent. Iontophoresis may be advantageously used to enhance or control the delivery rate of the active agent. The active agent may be stored in a reservoir such as a cavity. See e.g., U.S. Pat. No. 5,395,310. Alternatively, the active agent may be stored in a reservoir such as a porous structure or a gel. An ion exchange membrane may be positioned to serve as a polarity selective barrier between the active agent reservoir and the biological interface. The membrane, typically only permeable with respect to one particular type of ion (e.g., a charged active agent), prevents the back flux of the oppositely charged ions from the skin or mucous membrane.

Commercial acceptance of iontophoresis devices is dependent on a variety of factors, such as cost to manufacture, shelf life, stability during storage, efficiency and/or timeliness of active agent delivery, biological capability, and/or disposal issues. Commercial acceptance of iontophoresis devices is also dependent on their ability to hold and deliver drugs across various biological interfaces including, for example, tissue barriers. For example, it may be desirable to have novel approaches for packaging drugs in iontophoresis devices and delivering them.

The present disclosure is directed to overcome one or more of the shortcomings set forth above, and provide further related advantages.

### BRIEF SUMMARY

In one aspect, the present disclosure is directed to an iontophoretic drug delivery device for providing transdermal delivery of one or more therapeutic active agents to a biological interface. The iontophoretic drug delivery device includes an active electrode assembly including at least one active agent reservoir and at least one active electrode element operable to provide an electromotive force for driving one or more therapeutic agents from the at least one active agent reservoir to the biological interface.

In some embodiments, the at least one active agent reservoir includes a hydrogel matrix having a surface, the hydrogel matrix comprising at least one polymer selected from poly(amidoamines), poly(dimethylsiloxanes), poly(hydroxyethyl methacrylates), poly(N-isopropyl acrylamides), poly[1-vinyl-2-pyrrolidinone-co-(2-hydroxyethyl methacrylate)], poly(acrylamides), poly(acrylic acids), poly(methacrylic acids), poly(ethylene glycols), poly(ethylene glycol monomethacrylate), poly(methacryloyloxyethyl 5-amino salicylate), poly(methacrylic acid)-co-poly(ethylene glycol), poly(vinyl alcohols), and poly(vinyl-pyrrolidones), poly[methacrylic acid-co-polyethylene glycol monomethacrylate-co-methacryloyloxyethyl 5-amino salicylate], poly(2-hydroxyethyl methacrylate-co-methyl methacrylate), poly(acrylamides), poly(aminoproly methacrylamides), poly(N-(3-aminopropyl)methacrylamide), and poly(N,N-dimethy-2-aminoethyl methacrylate), or copolymers, block copolymers, graft copolymers, and heteropolymers thereof, or combinations thereof.

In another aspect, the present disclosure is directed to an iontophoretic drug delivery device for providing transdermal delivery of one or more therapeutic active agents to a biological interface. The iontophoretic drug delivery device includes an active electrode assembly including at least one active electrode element, at least one inner active agent reservoir, and an outermost active agent reservoir. The at least one inner active agent reservoir is positioned between the at least one active electrode element and the outermost active agent reservoir. The active electrode assembly is operable to provide an electrical potential.

The outermost active agent reservoir includes a hydrogel matrix having a surface. The hydrogel matrix may include at least one polymer selected from poly(amidoamines), poly(dimethylsiloxanes), poly(hydroxyethyl methacrylates), poly(N-isopropyl acrylamides), poly[1-vinyl-2-pyrrolidinone-co-(2-hydroxyethyl methacrylate)], poly(acrylamides), poly(acrylic acids), poly(methacrylic acids), poly(ethylene glycols), poly(ethylene glycol monomethacrylate), poly(methacryloyloxyethyl 5-amino salicylate), poly(methacrylic acid)-co-poly(ethylene glycol), poly(vinyl alcohols), and poly(vinyl-pyrrolidones), poly[methacrylic acid-co-polyethylene glycol monomethacrylate-co-methacryloyloxyethyl 5-amino salicylate], poly(2-hydroxyethyl methacrylate-co-methyl methacrylate), poly(acrylamides), poly(aminoproly methacrylamides), poly(N-(3-aminopropyl)methacrylamide), and poly(N,N-dimethy-2-aminoethyl methacrylate), or copolymers, block copolymers, graft copolymers, and heteropolymers thereof, or combinations thereof.

In yet another aspect, the present disclosure is directed to a method for transdermal administration of at least one cationic, anionic, or ionizable active agent. The method includes positioning an active electrode assembly and a counter electrode assembly of an iontophoretic delivery device on a biological interface of a subject. In some embodiments, the active electrode includes an active agent reservoir comprising a hydrogel matrix and at least one cationic, anionic, or ionizable active agent cached in the active agent reservoir.

The hydrogel matrix may include at least one polymer selected from poly(amidoamines), poly(dimethylsiloxanes), poly(hydroxyethyl methacrylates), poly(N-isopropyl acrylamides), poly[1-vinyl-2-pyrrolidinone-co-(2-hydroxyethyl methacrylate)], poly(acrylamides), poly(acrylic acids), poly(methacrylic acids), poly(ethylene glycols), poly(ethylene glycol monomethacrylate), poly(methacryloyloxyethyl 5-amino salicylate), poly(methacrylic acid)-co-poly(ethylene glycol), poly(vinyl alcohols), and poly(vinyl-pyrrolidones), poly[methacrylic acid-co-polyethylene glycol monomethacrylate-co-methacryloyloxyethyl 5-amino salicylate], poly(2-hydroxyethyl methacrylate-co-methyl methacrylate), poly(acrylamides), poly(aminoproly methacrylamides), poly(N-(3-aminopropyl)methacrylamide), and poly(N,N-dimethy-2-aminoethyl methacrylate), or copolymers, block copolymers, graft copolymers, and heteropolymers thereof, or combinations thereof.

The method further includes applying a sufficient amount of current to transport the at least one cationic, anionic, or ionizable active agent from the active agent reservoir, to the biological interface of the subject, and to administer a therapeutically effective amount of the at least one cationic, anionic, or ionizable active agent.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1A is a top, front view of a transdermal drug delivery system according to one illustrated embodiment.

FIG. 1B is a top, plan view of a transdermal drug delivery system according to one illustrated embodiment.

FIG. 2A is a schematic diagram of the iontophoresis device of FIGS. 1A and 1B comprising an active and counter electrode assemblies according to one illustrated embodiment.

FIG. 2B is a schematic diagram of the iontophoresis device of FIG. 2A positioned on a biological interface, with an optional outer release liner removed to expose the active agent, according to another illustrated embodiment.

FIG. 2C is a schematic diagram of the iontophoresis device comprising an active and counter electrode assemblies and a plurality of microneedles according to one illustrated embodiment.

FIG. 3A is a bottom, front view of a plurality of microneedles in the form of an array according to one illustrated embodiment.

FIG. 3B is a bottom, front view of a plurality of microneedles in the form of one or more arrays according to another illustrated embodiment.

FIG. 4 is a flow diagram of a method for transdermal administration of at least one cationic, anionic, or ionizable active agent according to one illustrated embodiment.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with iontophoresis devices including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “in another embodiment” means that a particular referent feature, structure, or characteristic described in connection with the embodiment is included in at least one embodiment. Thus, the appearance of the phrases “in one embodiment,” or “in an embodiment,” or “in another embodiment” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an iontophoresis device including “an electrode element” includes a single electrode element, or two or more electrode elements. It should also be noted that the term “or” is generally employed in its sense including “and/or” unless the content clearly dictates otherwise.

As used herein the term “membrane” means a boundary, a layer, barrier, or material, which may, or may not be permeable. The term “membrane” may further refer to an interface. Unless specified otherwise, membranes may take the form a solid, liquid, or gel, and may or may not have a distinct lattice, non cross-linked structure, or cross-linked structure.

As used herein the term “ion selective membrane” means a membrane that is substantially selective to ions, passing certain ions while blocking passage of other ions. An ion selective membrane, for example, may take the form of a charge selective membrane, or may take the form of a semi-permeable membrane.

As used herein the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH, and ACS also from Tokuyama Co., Ltd.

As used herein and in the claims, the term “bipolar membrane” means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules at a first rate, and some other molecules at a second rate different from the first. In yet further embodiments, the “semi-permeable membrane” may take the form of a selectively permeable membrane allowing only certain selective molecules to pass through it.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled macromolecules (e.g., cylindrical micelles). In some embodiments, a gel matrix may include hydrogels, organogels, and the like. Hydrogels refer to three-dimensional network of, for example, cross-linked hydrophilic polymers in the form of a gel and substantially composed of water. Hydrogels may have a net positive or negative charge, or may be neutral.

As used herein and in the claims, the term “reservoir” means any form of mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution.

As used herein and in the claims, the term “active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., a drug, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, an anti-tumor agent.

In some embodiments, the term “active agent” further refers to the active agent, as well as its pharmacologically active salts, pharmaceutically acceptable salts, prodrugs, metabolites, analogs, and the like. In some further embodiment, the active agent includes at least one ionic, cationic, ionizeable, and/or neutral therapeutic drug and/or pharmaceutical acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. Other active agents may be polarized or polarizable, that is exhibiting a polarity at one portion relative to another portion. For instance, an active agent having an amino group can typically take the form an ammonium salt in solid state and dissociates into a free ammonium ion (NH₄⁺) in an aqueous medium of appropriate pH.

The term “active agent” may also refer to electrically neutral agents, molecules, or compounds capable of being delivered via electro-osmotic flow. The electrically neutral agents are typically carried by the flow of, for example, a solvent during electrophoresis. Selection of the suitable active agents is therefore within the knowledge of one skilled in the relevant art.

In some embodiments, one or more active agents may be selected from analgesics, anesthetics, anesthetics vaccines, antibiotics, adjuvants, immunological adjuvants, immunogens, tolerogens, allergens, toll-like receptor agonists, toll-like receptor antagonists, immuno-adjuvants, immuno-modulators, immuno-response agents, immuno-stimulators, specific immuno-stimulators, non-specific immuno-stimulators, and immuno-suppressants, or combinations thereof.

Non-limiting examples of such active agents include lidocaine, articaine, and others of the -caine class; morphine, hydromorphone, fentanyl, oxycodone, hydrocodone, buprenorphine, methadone, and similar opioid agonists; sumatriptan succinate, zolmitriptan, naratriptan HCl, rizatriptan benzoate, almotriptan malate, frovatriptan succinate and other 5-hydroxytryptamine1 receptor subtype agonists; resiquimod, imiquidmod, and similar TLR 7 and 8 agonists and antagonists; domperidone, granisetron hydrochloride, ondansetron and such anti-emetic drugs; zolpidem tartrate and similar sleep inducing agents; L-dopa and other anti-Parkinson's medications; aripiprazole, olanzapine, quetiapine, risperidone, clozapine, and ziprasidone, as well as other neuroleptica; diabetes drugs such as exenatide; as well as peptides and proteins for treatment of obesity and other maladies.

Further non-limiting examples of anesthetic active agents or pain killers include ambucaine, amethocaine, isobutyl p-aminobenzoate, amolanone, amoxecaine, amylocalne, aptocaine, azacaine, bencaine, benoxinate, benzocaine, N,N-dimethylalanylbenzocaine, N,N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocalne, pramoxine, procaine (Novocaine®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and mixtures thereof.

As used herein and in the claims, the term “subject” generally refers to any host, animal, vertebrate, or invertebrate, and includes fish, mammals, amphibians, reptiles, birds, and particularly humans.

As used herein and in the claims, the term “agonist” refers to a compound that can combine with a receptor (e.g., a Toll-like receptor, and the like) to produce a cellular response. An agonist may be a ligand that directly binds to the receptor. Alternatively, an agonist may combine with a receptor indirectly by forming a complex with another molecule that directly binds the receptor, or otherwise resulting in the modification of a compound so that it directly binds to the receptor.

As used herein and in the claims, the term “antagonist” refers to a compound that can combine with a receptor (e.g., a Toll-like receptor, and the like) to inhibit a cellular response. An antagonist may be a ligand that directly binds to the receptor. Alternatively, an antagonist may combine with a receptor indirectly by forming a complex with another molecule that directly binds to the receptor, or otherwise results in the modification of a compound so that it directly binds to the receptor.

As used herein and in the claims, the term “effective amount” or “therapeutically effective amount” includes an amount effective at dosages and for periods of time necessary, to achieve the desired result. The effective amount of a composition containing a pharmaceutical agent may vary according to factors such as the disease state, age, gender, and weight of the subject.

As used herein and in the claims, the term “analgesic” refers to an agent that lessens, alleviates, reduces, relieves, or extinguishes a neural sensation in an area of a subject's body. In some embodiments, the neural sensation relates to pain, in other aspects the neural sensation relates to discomfort, itching, burning, irritation, tingling, “crawling,” tension, temperature fluctuations (such as fever), inflammation, aching, or other neural sensations.

As used herein and in the claims, the term “anesthetic” refers to an agent that produces a reversible loss of sensation in an area of a subject's body. In some embodiments, the anesthetic is considered to be a “local anesthetic” in that it produces a loss of sensation only in one particular area of a subject's body.

As one skilled in the relevant art would recognize, some agents may act as both an analgesic and an anesthetic, depending on the circumstances and other variables including but not limited to dosage, method of delivery, medical condition or treatment, and an individual subject's genetic makeup. Additionally, agents that are typically used for other purposes may possess local anesthetic or membrane stabilizing properties under certain circumstances or under particular conditions.

As used herein and in the claims, the term “immunogen” refers to any agent that elicits an immune response. Examples of an immunogen include, but are not limited to natural or synthetic (including modified) peptides, proteins, lipids, oligonucleotides (RNA, DNA, etc.), chemicals, or other agents.

As used herein and in the claims, the term “allergen” refers to any agent that elicits an allergic response. Some examples of allergens include but are not limited to chemicals and plants, drugs (such as antibiotics, serums), foods (such as milk, wheat, eggs, etc), bacteria, viruses, other parasites, inhalants (dust, pollen, perfume, smoke), and/or physical agents (heat, light, friction, radiation). As used herein, an allergen may be an immunogen.

As used herein and in the claims, the term “adjuvant” and any derivations thereof, refers to an agent that modifies the effect of another agent while having few, if any, direct effect when given by itself. For example, an adjuvant may increase the potency or efficacy of a pharmaceutical, or an adjuvant may alter or affect an immune response.

As used herein and in the claims, the terms “vehicle,” “carrier,” “pharmaceutically vehicle,” “pharmaceutically carrier,” “pharmaceutically acceptable vehicle,” or “pharmaceutically acceptable carrier” may be used interchangeably, and refer to pharmaceutically acceptable solid or liquid, diluting or encapsulating, filling or carrying agents, which are usually employed in pharmaceutical industry for making pharmaceutical compositions. Examples of vehicles include any liquid, gel, salve, cream, solvent, diluent, fluid ointment base, vesicle, liposomes, nisomes, ethasomes, transfersomes, virosomes, cyclic oligosaccharides, non ionic surfactant vesicles, phospholipid surfactant vesicles, micelle, and the like, that is suitable for use in contacting a subject.

In some embodiments, the pharmaceutical vehicle may refer to a composition that includes and/or delivers a pharmacologically active agent, but is generally considered to be otherwise pharmacologically inactive. In some other embodiments, the pharmaceutical vehicle may have some therapeutic effect when applied to a site such as a mucous membrane or skin, by providing, for example, protection to the site of application from conditions such as injury, further injury, or exposure to elements. Accordingly, in some embodiments, the pharmaceutical vehicle may be used for protection without a pharmacological agent in the formulation.

As used herein and in the claims, the term “functional group” generally refers to a chemical group that confers special properties or particular functions to an article (e.g., a surface, a molecule, a polymer, a substance, a particle, nanoparticle, and the like). Among the chemical groups, examples include an atom, an arrangement of atoms, an associated group of atoms, molecules, moieties, and that like, that confer certain characteristic properties on the article comprising the functional groups. Exemplary characteristic properties and/or functions include chemical properties, chemically reactive properties, association properties, electrostatic interaction properties, bonding properties, biocompatible properties, and the like. In some embodiments, the functional groups include one or more nonpolar, hydrophilic, hydrophobic, organophilic, lipophilic, lipophobic, acidic, basic, neutral, functional groups, and the like.

The headings provided herein are for convenience only and do not interpret the scope or meaning of the embodiments.

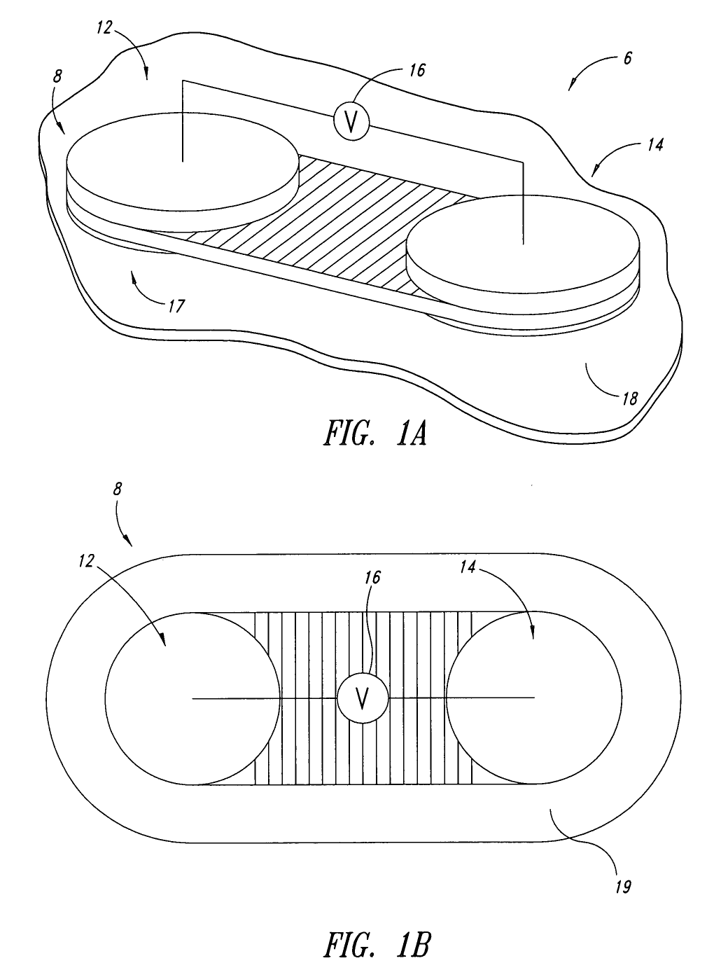

FIGS. 1A and 1B show an exemplary iontophoretic drug delivery system 6 for delivering of one or more active agents to a subject. The system 6 includes an iontophoresis device 8 including active and counter electrode assemblies 12, 14, respectively, and a power source 16. The active and counter electrode assemblies 12, 14, are electrically coupleable to the power source 16 to supply an active agent contained in the active electrode assembly 12, via iontophoresis, to a biological interface 18 (e.g., a portion of skin or mucous membrane). The iontophoresis device 8 may optionally include a non-conductive biocompatible backing 19. In some embodiments, the non-conductive biocompatible backing 19 encases the iontophoresis devices 8. In some other embodiments, the non-conductive biocompatible backing 19 physically couples the iontophoresis device 8 to the biological interface 18 of the subject. In some embodiments, the system 6 is configured for providing transdermal delivery of one or more therapeutic active agents to a biological interface of a subject and inducing analgesia or aesthesia in the subject for a limited period of time.

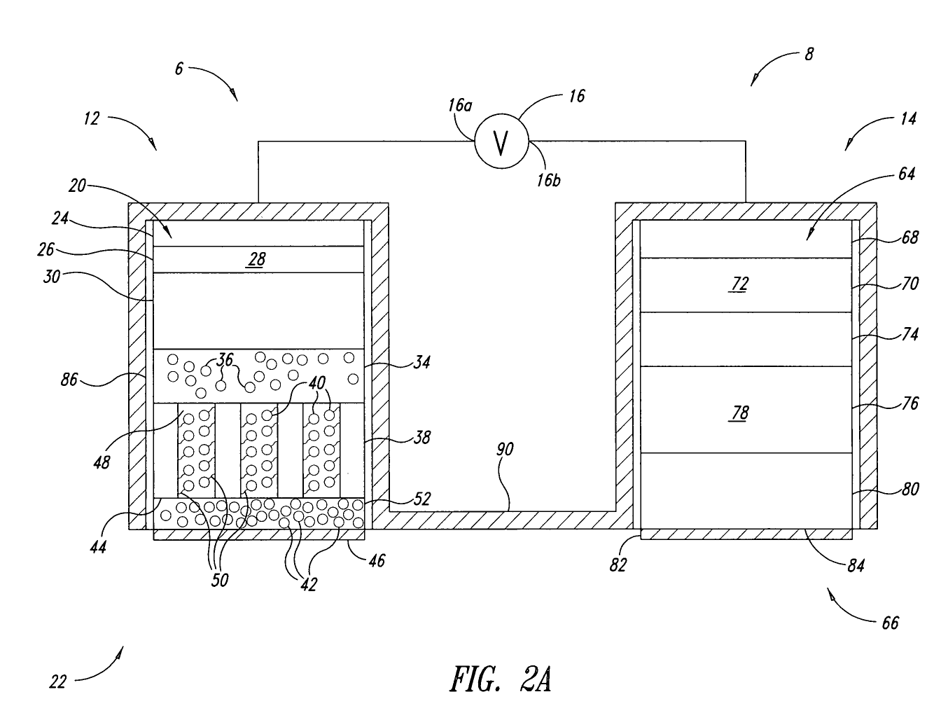

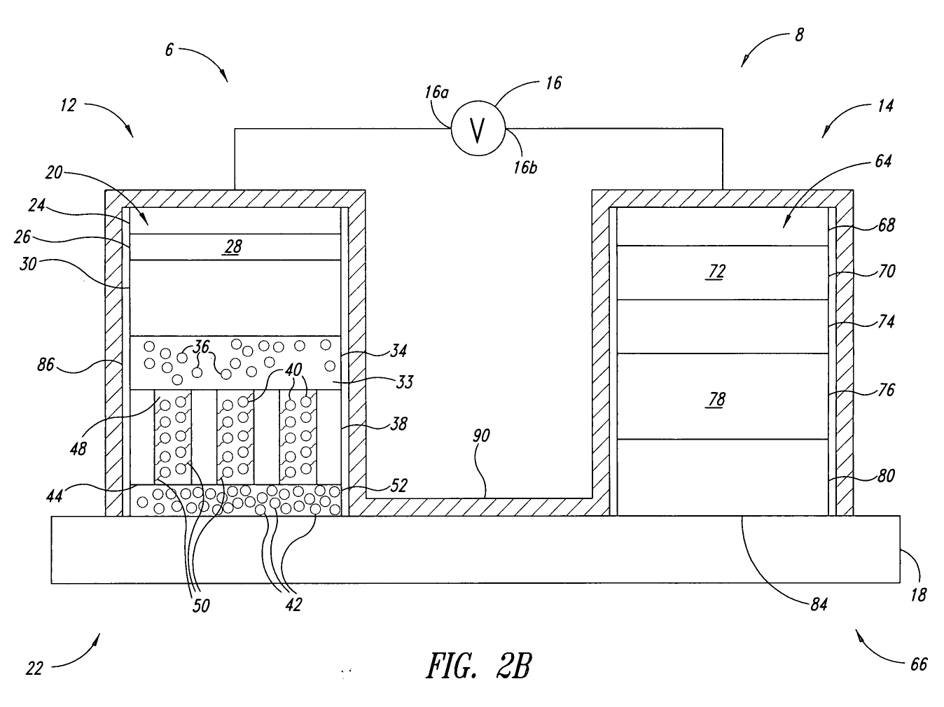

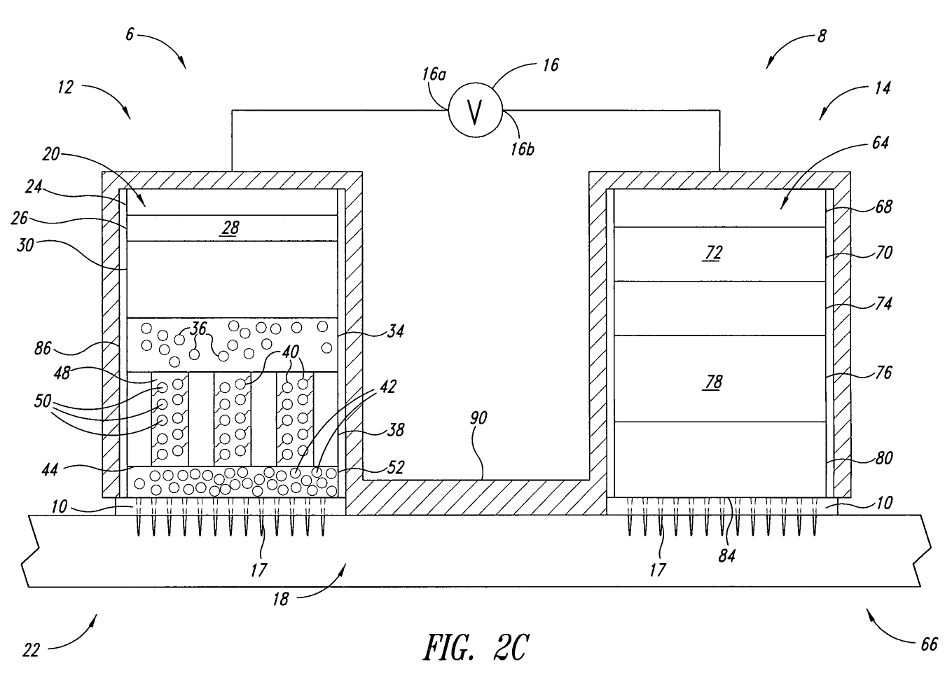

As shown in FIGS. 2A and 2B, the active electrode assembly 12 may further comprise, from an interior 20 to an exterior 22 of the active electrode assembly 12: an active electrode element 24, an electrolyte reservoir 26 storing an electrolyte 28, an inner ion selective membrane 30, one or more inner active agent reservoirs 34, storing one or more active agents 36, an optional outermost ion selective membrane 38 that optionally caches additional active agents 40, and an optional further active agent 42 carried by an outer surface 44 of the outermost ion selective membrane 38. The active electrode assembly 12 may further comprise an optional outer release liner 46.

The at least one active agent reservoir 34 is loadable with a vehicle for transporting, delivering, encapsulating, and/or carrying the one or more active agents 36, 40, 42. In some embodiments, the vehicle may take the form of a hydrogel matrix. Examples of vehicles include degradable or non-degradable polymers, hydrogels, organogels, liposomes, nisomes, ethasomes, transfersomes, virosomes, cyclic oligosaccharides, non-ionic surfactant vesicles, phospholipid surfactant vesicles, micelles, microspheres, creams, emulsions, lotions, pastes, gels, ointments, organogel, and the like, as well as any matrix that allows for transport of an agent across the skin or mucous membranes of a subject. In at least one embodiment, the vehicle allows for controlled release formulations of the compositions disclosed herein.

As one skilled in the relevant art would appreciate, pharmaceutical formulations employed in forming, for example, pharmaceutically acceptable vehicles for transporting one or more active agents 36, 40, 42 will be readily understood in the art. For example, ointments may be semisolid preparations based on petrolatum or other petroleum derivatives. Emulsions may be water in oil or oil in water and include, for example, cetyl alcohol, gylceryl monostearate, lanolin and steric acid, and may also contain polyethylene glycols. Creams may be viscous liquids or semisolid emulsions of oil in water or water in oil. Gels may be semisolid suspensions of molecules including organic macromolecules as well as an aqueous, alcohol, and/or oil phase. Examples of such organic macromolecules include gelling agents (e.g., carboxypolyalkylenes, and the like), hydrophilic polymers (e.g., polyethylene oxides, polyoxyethylene-polyoxypropylene copolymers, polyvinylalcohols, and the like) cellulosic polymers (e.g., hydroxypropyl cellulose, hydroxyethyl cellulose, hydroxypropyl methylcellulose, hydroxypropyl methylcellulose, phthalate, methyl cellulose, and the like), tragacanth or xanthan gums, sodium alginate, gelatin, and the like, or combination thereof.

In some embodiments, the one or more active agent reservoirs 34 include a hydrogel matrix having a surface. In some embodiments, the hydrogel matrix includes at least one polymer selected from poly(amidoamines), poly(dimethylsiloxanes), poly(hydroxyethyl methacrylates), poly(N-isopropyl acrylamides), poly[1-vinyl-2-pyrrolidinone-co-(2-hydroxyethyl methacrylate)], poly(acrylamides), poly(acrylic acids), poly(methacrylic acids), poly(ethylene glycols), poly(ethylene glycol monomethacrylate), poly(methacryloyloxyethyl 5-amino salicylate), poly(methacrylic acid)-co-poly(ethylene glycol), poly(vinyl alcohols), and poly(vinyl-pyrrolidones), poly[methacrylic acid-co-polyethylene glycol monomethacrylate-co-methacryloyloxyethyl 5-amino salicylate], poly(2-hydroxyethyl methacrylate-co-methyl methacrylate), poly(acrylamides), poly(aminoproly methacrylamides), poly(N-(3-aminopropyl)methacrylamide), and poly(N,N-dimethy-2-aminoethyl methacrylate), or copolymers, block copolymers, graft copolymers, and heteropolymers thereof, or combinations thereof.

Protocols for forming hydrogel matrix and/or pharmaceutically acceptable vehicles in the form of hydrogels matrix are well known in the relevant art. The manner of treatment is dependent on, for example, the nature of the chemical compound to be synthesized and the nature and composition of the surface. See, for example, Segura et al., Crosslinked Hyaluronic Acid Hydrogels: a Strategy to Functionalize and Pattern” 26(4), pp. 359-71 (2005); Dvaran et al., “Synthesis and Characterization of Methacrylic Derivatives of 5-Amino Salicylic Acid with pH-Sensitive Swelling Properties” MPS PharmSciTech, 2(4), article 29 (2001).

Protocols for modifying polymers are well known in the relevant art and include, for example, modifying one or more functional groups of the at least one polymer, modifying the backbone of the at least one polymer, modifying one or more polymeric units of the least one polymer, and the like. See, for example, Barbu et al., “Polymeric Materials for Ophthalmic Drug Delivery: Trends and Perspectives” J. Mater. Chem. 16, pp. 3439-3443 (2006); Yadavalli et al., “Microfabricated protein-containing poly(ethylene glycol) hydrogel arrays for biosensing” Sensors and Actuators B-Chemical, 97:290-297 (2004); “Persistent Interactions Between Hydroxylated Nanoballs and Atactic poly(2-Hydroxyethyl Methacrylate) (PHEMA)” Chemical Communications, pp. 3277-3279, (2005); as well as U.S. Pat. Nos. 5,770,627, and 5,804,318.

Further examples of backbone modification include forming graft copolymers and/or block copolymers, as well as introducing functional groups onto, for example, the hydroxyethyl methacrylate backbone, the poly(vinyl alcohol) backbone, and the like of the hydrogel matrix.

In some embodiments, one or more polymeric units of the least one polymer are modified with one or more groups selected from charge functional groups, hydrophobic functional groups, hydrophilic functional groups, chemically reactive functional groups, organofunctional group, and bio-compatible groups. In some embodiments, at least a portion of a polymeric backbone of the least one polymer has been modified with one or more groups selected from carboxylate groups, sulfonate groups, amine groups, quaternary amine groups, alkoxy amines, aspartic acids, iminodiacetic acids, and glutamic acids. In yet some other embodiments, one or more polymeric units of the least one polymer are modified with one or more groups selected from carboxylate groups, sulfonate groups, amine groups, quaternary amine groups, alkoxy amines, aspartic acids, iminodiacetic acids, and glutamic acids. The least one polymer may be selected from backbone-modified hydroxyethyl methacrylate polymers having one or more backbone units modified with one or more groups selected from carboxylate groups, sulfonate groups, amine groups, quaternary amine groups, alkoxy amines, aspartic acids, iminodiacetic acids, and glutamic acids.

In some embodiments, one or more polymeric units of the least one polymer may be modified with one or more polymeric units selected to impart one or more of properties to the surface of the hydrogel matrix including nonpolar, hydrophilic, hydrophobic, organophilic, lipophilic, lipophobic, acidic, basic, neutral, properties, increased or decreased permeability, and the like, and/or combinations thereof.

In some embodiments, the at least one active agent reservoir 34 may further include a therapeutically effective amount of one or more active agents 36, 40, 42 cached in the at least one active agent reservoir 34 comprising the hydrogel matrix. In some embodiments, the one or more active agents 36, 40, 42 are selected from cationic, anionic, ionizable, or neutral active agents.

In some embodiments, the one or more active agents 36, 40, 42 may be capable of increasing, decreasing, altering, initiating, and/or extinguishing a biological response. As one skilled in the relevant art would recognize, dosing of a particular active agent may depend on the specific medical condition or indication, method of treatment or delivery, the subject's age, the subject's weight, the subject's gender, the subject's genetic makeup, the subject's overall health, as well as other factors. In some embodiments, the iontophoresis delivery device 8 may be configured to provide controlled-delivery or sustained-delivery of the pharmaceutically acceptable vehicle including one or more active agents 36, 40, 42.

Examples of the one or more active agents 36, 40, 42 include one or more immuno-adjuvants, immuno-modulators, immuno-response agents, immuno-stimulators, specific immuno-stimulators, non-specific immuno-stimulators, and immuno-suppressants, vaccines, agonists, antagonist, opioid agonist, opioid antagonist, antigens, adjuvants, immunological adjuvants, immunogens, tolerogens, allergens, toll-like receptor agonists, toll-like receptor antagonists, and the like, or combinations thereof.

Further examples of the at one or more active agents 36, 40, 42 include at least one analgesic or anesthetic active agent selected from alfentanil, codeine, COX-2 inhibitors, opiates, opioid agonist, opioid antagonist, diamorphine, fentanyl, meperidine, methadone, morphine morphinomimetics, naloxone, nonsteroidal anti-inflammatory drugs (NSAIDs), oxycodone, remifentanil, sufentanil, and tricyclic antidepressants, or combinations thereof. In some embodiments, the one or more active agents 36, 40, 42 are selected from analgesics, anesthetics, or combinations thereof.

As one skilled in the relevant art would recognize, multiple and various analgesics may be employed as active agents 36, 40, 42. Suitable analgesics include, for example, non-steroidal anti-inflammatory compounds, natural and synthetic opiates or opioids, morphine, Demorol® (meperidine), Dilaudid® (hydromorphone), Sublimaze® (fentanyl), acetaminophen, Darvocet® (propoxyphene and acetaminophen), codeine, naproxen, aspirin, ibuprofen, Vicodin® (hydrocodone bitartrate and acetaminophen), Percocet® (acetaminophen and oxycodone), Vicoprofen® (hydrocodone and ibuprofen), Ultram® (tramadol), Dolphine® (methadone), OxyContin® (oxycodone), COX-2 inhibitors (such as celecoxib and rofecoxib), prednisone, etodolac, nabumetone, indomethacin, sulindac, tolmetin sodium, ketorolac tromethamine, trisalicylate, diflunisal, salsalate, sodium salicylate, sodium thiosalicylate, flurbiprofen, fenoprofen, ketoprofen, oxaprozin, piroxicam, isoxicam, meclofenamate, diclofenac, epinephrine, benzodiazepines, cannabinoids, caffeine, hydroxyzine, and the like, or any combination thereof.

Some analgesics may function, for example, by interfering with nerve reception or response, by interfering with cell receptors, by interfering with production of a cellular component, by interfering with regulation of a particular gene transcription or protein translation, by interfering with protein excretion or secretion, by interfering with cellular membrane components, any combination thereof, or by other means. Some local anesthetics may cause reversible loss of sensation in an area of a subject's body by interrupting nerve impulses or responses, by influencing membrane variations, by influencing production of cellular components, by interrupting nerve conductance, by interrupting gene transcription or protein translation, by interfering with protein secretion or excretion, any combination thereof, or by other means. Some topical anesthetics may have a rapid onset of action (for example, approximately in 10 minutes or less, approximately in 5 minutes or less, etc.), and/or may have a moderate duration of action (approximately 30-60 minutes, or more).

As one skilled in the relevant art would recognize that multiple and various anesthetics could be employed. For example, several suitable local anesthetic agents consist of an aromatic ring linked by a carbonyl-containing moiety through a carbon chain to a substituted amino group, including esters, amides, quinolones, and the like. In certain embodiments, the anesthetic may be present in the composition as a free base to promote penetration of the agent through the skin or mucosal surface. Examples of some other anesthetics include centbucridine, tetracaine, Novocaine® (procaine), ambucaine, amolanone, amylcaine, benoxinate, betoxycaine, carticaine, chloroprocaine, cocaethylene, cyclomethycaine, butethamine, butoxycaine, carticaine, dibucaine, dimethisoquin, dimethocaine, diperodon, dyclonine, ecogonidine, ecognine, euprocin, fenalcomine, formocaine, hexylcaine, hydroxyteteracaine, leucinocaine, levoxadrol, metabutoxycaine, methyl chloride, myrtecaine, butamben, bupivicaine, mepivacaine, beta-adrenoceptor antagonists, opioid analgesics, butanilicaine, ethyl aminobenzoate, fomocine, hydroxyprocaine, isobutyl p-aminobenzoate, naepaine, octacaine, orthocaine, oxethazaine, parenthoxycaine, phenacine, phenol, piperocaine, polidocanol, pramoxine, prilocalne, propanocaine, proparacaine, propipocaine, pseudococaine, pyrrocaine, salicyl alcohol, parethyoxycaine, piridocaine, risocaine, tolycaine, trimecaine, tetracaine, anticonvulsants, antihistamines, articaine, cocaine, procaine, amethocaine, chloroprocaine, Lidocaine® (xylocaine), marcaine, chloroprocaine, etidocaine, prilocalne, lignocaine, benzocaine, zolamine, ropivacaine, dibucaine, and the like or pharmaceutically acceptable salt thereof, or mixtures thereof.

In some embodiments, the hydrogel matrix may further comprise a therapeutically effective amount of one or more immunity agents. An immunity agent may be capable of increasing, decreasing, altering, initiating or extinguishing an immune response. As one skilled in the relevant art would recognize, dosing of a particular active agent may depend on the specific medical condition or indication, method of treatment or delivery, the subject's age, the subject's weight, the subject's gender, the subject's genetic makeup, the subject's overall health, as well as other factors. In at least one embodiment of the pharmaceutical composition, the immunity agent is capable of functioning as an adjuvant. In certain embodiments, the immunity agent is a Toll-like receptor agonist or antagonist.

Toll-like receptors may initiate immune responses by, among other things, activating dendritic cells. For example, some toll-like receptors belong to a family of receptors called pattern-recognition receptors, which may be activated upon recognition of “Pathogen-Associated Molecular Patterns” or PAMPs. PAMPs are molecular patterns common to many pathogens. Examples of some PAMPs include, but are not limited to, cell wall constituents such as lipopolysaccharide, peptidoglycan, lipoteichoic acid, lipoarabinomannan, single or double stranded RNA, and unmethylated CpG DNA.

A number of Toll-like receptors have been identified in mammals and are included in various embodiments of the present disclosure. For example, TLR1, TLR2, TLR3, TLR4, TLR5, TLR6, TLR7, TLR8, TLR9, TLR10, TLR11, TLR12 (mouse only), TLR13 (mouse only), have all been identified in mice and/or humans. Agonists or antagonists to any and/or all of these Toll-like receptors and others not yet identified may be included in various embodiments.

Stimulation of Toll-like receptors by pathogens results in expression of multiple immune response genes, including NF-κB, mitogen activated protein kinases p38, Jun-N-terminal kinase, and the interferon pathway.

Some examples of Toll-like receptor agonists include, but are not limited to, isatoribine, natural or synthetic lipopeptides (e.g., Pam3CSK4, also called palmitoyl-3-cysteine-serine-lysine-4), bacteria or fragments of bacteria, including heat killed L. Monocytogenes (HLKM) and Flagellin S. typhimurium, natural or synthetic RNA (e.g., Poly(I:C) and ssRNA40), natural or synthetic lipopolysaccharides (e.g., LPS E. coli K12), natural or synthetic oligonucleotides or oligonucleotide analogues (e.g., imiquimod and ODN2006), and the like. Additionally, Toll-like receptor agonists that have not yet been identified may also be included in various embodiments.

Some examples of Toll-like receptor antagonists include, but are not limited to natural or synthetic lipopolysaccharides (e.g., LPS-PG, isolated from P. gingivalis; and LPS-EK msbB, isolated from E. coli K12 msbB), or natural or synthetic oligonucleotides (e.g., ODN 2088 (suppressive ODN, mouse specific); and ODN TTAGGG (suppressive ODN, human specific)), and the like. Additionally, Toll-like receptor antagonists that have not yet been identified may also be included in various embodiments.

One skilled in the relevant art would recognize that some or all of the compositions herein described are suitable for pharmaceutical compositions. At least some embodiments include a pharmaceutical composition comprising a hydrogel matrix and an effective amount of an active agent in the form of an active immunity agent. In at least one embodiment of the pharmaceutical composition, the immunity agent is a Toll-like receptor agonist. In at least one embodiment of the pharmaceutical composition, the immunity agent is a Toll-like receptor antagonist. In at least one embodiment of the pharmaceutical composition, the vehicle allows for controlled release of the immunity agent.

In some embodiments, the hydrogel matrix may further comprise an adjuvant. Multiple different adjuvants are known in the art, and are described, for example, in William E. Paul “Fundamental Immunology” Lippincot Williams & Wilkins (5th ed. 2003) and Janeway et al. “Immunobiology” Elsevier Science Health Science div (6th ed., 2004).

In some embodiments, the adjuvant alters the immune response of the biological factor administered in conjunction with the adjuvant. In at least one aspect, the adjuvant alters the potency of an immune response. In at least one aspect, the adjuvant alters the type of immune response to the biological factor. In at least one aspect, the adjuvant increases the potency of an immune response. In at least one aspect, the adjuvant decreases the potency of an immune response. In at least one aspect, the adjuvant alters both the potency and the type of immune response to the biological factor. The biological factor may be injected, orally administered, iontophoretically administered or otherwise introduced to a subject.

As used herein and in the claims, “in conjunction with” and any derivations thereof, refers to administration of the adjuvant simultaneously with, prior to, or subsequent to administration of the biological factor. In at least one embodiment, the adjuvant is administered simultaneously with the biological factor. In at least one embodiment, the adjuvant is administered prior to the biological factor. In at least one embodiment, the adjuvant is administered subsequent to the biological factor.

Some adjuvants may alter an immune response to a biological factor administered in conjunction with the adjuvant, while not altering an immune response when the adjuvant is administered alone. Examples of adjuvants that may act directly or indirectly on an immune system or on hematopoeitic cells and/or components include antigen presenting cells, such as dendritic cells and Langerhans cells, and/or other components such as lymphocytes (T cells, B cells, etc.), monocytes, macrophages, neutrophils, eosinophils, red blood cells, platelets, basophils, and/or supportive cells (stromal cells, stem cells, tissue cells), or any combination thereof. In addition, an adjuvant may alter production or degradation of chemicals associated with immune responses, including cytokines, nitric oxide, heat shock proteins, vasodilators, vasoconstrictors, neurotransmitters, other neurotrophic factors, hemoglobin, and any other biological chemical that may affect an immune system component.

In some embodiments, the hydrogel matrix may further comprise one or more additional ingredients, such as one or more thickening agents, medicinal agents, growth factors, immune system agents, wound-healing factors, peptidomimetics, proteins or peptides, carbohydrates, bioadhesive polymers, preservatives, inert carriers, caffeine or other stimulants (such as epinephrine, norepinephrine, adrenaline, etc.), lipid absorbents, chelating agents, buffers, anti-fading agents, stabilizers, moisture absorbents, vitamins, UV blockers, humectants, cleansers, colloidal meals, abrasives, herbal extracts, phytochemicals, fragrances, colorants or dyes, film-forming materials, analgesics, etc. A single excipient may perform multiple functions or a single function. One skilled in the relevant art will readily be able to identify and choose any such excipients based on the desired physical and chemical properties of the final formulation.

Examples of some commonly used thickening agents include, but are not limited to, cellulose, hydroxypropyl cellulose, methyl cellulose, polyethylene glycol, sodium carboxymethyl cellulose, polyethylene oxide, xanthan gum, guar gum, agar, carrageenan gum, gelatin, karaya, pectin, locust-bean gum, aliginic acid, bentonite carbomer, povidone, tragacanth, and the like, or any combination thereof.

One skilled in the relevant art would also readily be able to identify and choose any optional medicinal agents or their pharmaceutically acceptable salts, based on the desired effect for the final formulation. Examples of medicinal agents include, but are not limited to, antifungal compositions (e.g., ciclopirox, triacetin, nystatin, tolnaftate, miconizole, clortrimazole, and the like), antibiotics (gentamicin, polymyxin, bacitracin, erythromycin, and the like), antiseptics (iodine, povidine, benzoic acid, benzyol peroxide, hydrogen peroxide, and the like), and anti-inflammatory compositions (e.g., hydrocortisone, prednisone, dexamethasone, and the like), or any combination thereof.

One skilled in the relevant art would also readily identify and choose any optional bioadhesive polymers that may be useful for hydrating the skin, ensuring surface contact and/or increasing pharmaceutical delivery. Some examples of bioadhesive polymers include, but are not limited to pectin, alginic acid, chitosan, hyaluronic acid, polysorbates, polyethyleneglycol, oligosaccharides, polysaccharides, cellulose esters, cellulose ethers, modified cellulose polymers, polyether polymers and oligomers, polyether compounds (block copolymers of ethylene oxide and propylene oxide) polyacrylamide, poly vinyl pyrrolidone, polymethacrylic acid, polyacrylic acid, or any combination thereof.

One skilled in the relevant art would recognize that the teachings herein may be utilized with wounded or intact skin, or on mucous membranes, including but not limited to oral, bronchial, vaginal, rectal, uterine, urethral, optic, ophthalmologic, pleural, nasal, or the like.

In some embodiments, the hydrogel matrix may further comprise at least a therapeutically effective amount of a first active agent and a therapeutically effective amount of a second active agent, the second active agent different from the first active agent, the first and the second active agents stored in the at least one active agent reservoir 34 of the iontophoresis delivery device 8.

In some embodiments, the first active agent is selected from an analgesic and the second active agent is selected from an antihistamine drug. In some other embodiments, the first active agent is selected from an analgesic and the second active agent is selected from a steroid. In some other embodiments, the first active agent is selected from an analgesic and the second active agent is selected from a vasoconstrictor drug. The hydrogel matrix comprising the first and the second active agents may be stored in the at least one active agent reservoir.

In some embodiments, the one or more therapeutic active agents 36, 40, 42 are selected form cationic active agents, and one or more polymeric units of the at least on polymer are modified with negatively charged functional groups. In some embodiments, a substantial portion of the one or more therapeutic active agents are carried by a portion of the surface of the hydrogel matrix, prior to use, in the absence of an electromotive force or current.

In some embodiments, the iontophoresis device 8 is operable to deliver one or more active agents 36, 42, 44 to a biological interface 18 such as skin or mucous membranes. The iontophoresis device 8 includes a hydrogel matrix containing ion-exchange functionalities to bind ionized drug and/or counter-ions creating a reservoir 34 with ion-exchange and exclusion properties similar to that of an ion-exchange membrane. One aspect includes derivatives of the hydrogel backbone. In some embodiments, the hydrogels include one or more polymers selected from polyvinyl alcohols, hydroxyethyl methacrylates, and the like. In some embodiments, the hydrogels may also include derivatives selected from carboxylate, cufonate, amine, and quaternary amine groups. Derivatives may contain strong and/or week ionic functionalities. In some further embodiments, derivatives of the hydrogel backbone may be incorporated with non-derivative backbone hydrogels into the hydrogel matrices.

In some embodiments, the outermost ion selective membrane 38 takes the form of a hydrogel matrix having ion-exchange functionalities to bind ionic and/or ionized drug and/or counter-ions creating a reservoir with ion exchange and exclusion properties similar to that of an ion-exchange membrane. Some further embodiments include derivatives of the hydrogel backbone. In some embodiments, the hydrogel includes on or more polymers selected from polyvinyl alcohol (PVA) and/or hydroxyethyl methacrylate (HEMA). In some other embodiments, the hydrogels may be modified with on or more derivatives selected from carboxylate, sulfonate, amine, and quaternary amine groups. Derivative may contain strong and/or week ionic functionalities. In some embodiments, derivatives of the hydrogel backbone may be incorporated with non-derivatized backbone hydrogels.

The advantage of using a hydrogel matrix in addition to or in place of an ion-exchange membrane is that the use of a hydrogel matrix or reservoir 34 enables the iontophoresis device 8 to incorporate additional active agents in a bound state, while retaining the ion-exchange properties described above.

A wide variety of charged functional groups of either charge can be incorporated in varied degrees of density into the hydrogel matrices. U.S. Pat. Nos. 4,731,049; 4,915,685; 4,927,048; 5,057,072; 5,084,008; 5,395,310; 5,871,460 and 6,049,733 describe additional modifications of the active and counter electrode systems 12, 14 of an iontophoretic device 8 to incorporate ion-exchange membranes to inhibit counter ion flow and enhancement of delivery of the desired drug and are hereby incorporated in their entirety by reference.

Hydrogel matrices are made in part by using various types of polymers. Polymers are long, chain molecules made of regular repeating polymeric units/patterns of building blocks (monomers). Naturally occurring polymers are common in nature and have been used as wound treatments (e.g., various forms of collagen). Many industrial polymers use a single monomer or combine two monomers into A-A-A or A-B-A structures, respectively. Purely synthetic hydrogels used in medical applications are frequently made from polyvinyl pyrrolidone, polyacrylamide, or polyethylene oxide. The structure of polyethylene oxide, which is contained in VIGILON® (CR Bard, Covington, Ga.) is shown below:
—(CH₂—CH₂—O—CH₂—CH₂—O—CH₂—CH₂—O)—

Noncovalent interactions between the adjacent polymer molecules enable the strands to stick to each other, particularly if the monomers contain aromatic rings, and this effect can lend strength to devices constructed from the polymer. To impart further structural integrity to the polymer, polymer molecules are covalently cross-linked using, for example, free radical reactions to activate side chains that protrude from the monomers. While this cross-linking can be accomplished chemically, the least expensive and most uniform result is achieved by irradiating the uncrosslinked polymer with ultraviolet light or electron beam.

In some embodiments, the active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42 may sufficiently cover the outer surface 44 and/or be of sufficient thickness so as to form a distinct layer 52. In other embodiments, the further active agent 42 may not be sufficient in volume, thickness, or coverage as to constitute a layer in a conventional sense of such term.

The active agent 42 may be deposited in a variety of highly concentrated forms such as, for example, solid form, nearly saturated solution form or gel form. If in solid form, a source of hydration may be provided, either integrated into the active electrode assembly 12, or applied from the exterior thereof just prior to use.

Referring to FIGS. 2A and 2B, the active electrode assembly 12 of the iontophoretic delivery device 8 may further comprise an optional inner sealing liner (not shown) between two layers of the active electrode assembly 12, for example, between the inner ion selective membrane 30 and the inner active agent reservoir 34. The inner sealing liner, if present, would be removed prior to application of the iontophoretic device to the biological surface 18. Each of the above elements or structures will be discussed in detail below.

In some embodiments, the system 6 takes the form of a self-contained iontophoretic drug delivery system. The system 6 includes at least one active agent reservoir 34, an active electrode assembly 12 including at least one active electrode element 24, and a power source 16. The at least one active agent reservoir 34 includes a pharmaceutical composition for inducing analgesia or anesthesia in the subject. The pharmaceutical composition for inducing analgesia or anesthesia in the subject may include at least one algesic or anesthetic active agent in combination with at least one opioid antagonist.

The active electrode element 24 is electrically coupled to a first pole 16a of the power source 16 and positioned in the active electrode assembly 12 to apply an electromotive force to transport the active agent 36, 40, 42 via various other components of the active electrode assembly 12. Under ordinary use conditions, the magnitude of the applied electromotive force is generally that required to deliver the one or more active agents according to a therapeutic effective dosage protocol. In some embodiments, the magnitude is selected such that it meets or exceeds the ordinary use operating electrochemical potential of the iontophoresis delivery device 8. The at least one active electrode element 24 is operable to provide an electromotive force for driving the pharmaceutical composition (comprising the at least one algesic or anesthetic active agent in combination with the at least one opioid antagonist) for inducing analgesia or anesthesia in the subject from the at least one active agent reservoir 34, to the biological interface 18 of the subject.

The active electrode element 24 may take a variety of forms. In one embodiment, the active electrode element 24 may advantageously take the form of a carbon-based active electrode element. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004. The carbon-based electrodes are inert electrodes in that they do not themselves undergo or participate in electrochemical reactions. Thus, an inert electrode distributes current through the oxidation or reduction of a chemical species capable of accepting or donating an electron at the potential applied to the system, (e.g., generating ions by either reduction or oxidation of water). Additional examples of inert electrodes include stainless steel, gold, platinum, capacitive carbon, or graphite.

Alternatively, an active electrode of sacrificial conductive material, such as a chemical compound or amalgam, may also be used. A sacrificial electrode does not cause electrolysis of water, but would itself be oxidized or reduced. Typically, for an anode a metal/metal salt may be employed. In such case, the metal would oxidize to metal ions, which would then be precipitated as an insoluble salt. An example of such anode includes an Ag/AgCl electrode. The reverse reaction takes place at the cathode in which the metal ion is reduced and the corresponding anion is released from the surface of the electrode.

The electrolyte reservoir 26 may take a variety of forms including any structure capable of retaining electrolyte 28, and in some embodiments may even be the electrolyte 28 itself, for example, where the electrolyte 28 is in a gel, semi-solid or solid form. For example, the electrolyte reservoir 26 may take the form of a pouch or other receptacle, a membrane with pores, cavities, or interstices, particularly where the electrolyte 28 is a liquid.

In one embodiment, the electrolyte 28 comprises ionic or ionizable components in an aqueous medium, which can act to conduct current towards or away from the active electrode element. Suitable electrolytes include, for example, aqueous solutions of salts. Preferably, the electrolyte 28 includes salts of physiological ions, such as, sodium, potassium, chloride, and phosphate. In some embodiments, the one or more electrolyte reservoirs 24 including an electrolyte 28 comprising at least one biologically compatible anti-oxidant selected from ascorbate, fumarate, lactate, and malate, or salts thereof.

Once an electrical potential is applied, when an inert electrode element is in use, water is electrolyzed at both the active and counter electrode assemblies. In certain embodiments, such as when the active electrode assembly is an anode, water is oxidized. As a result, oxygen is removed from water while protons (H+) are produced. In one embodiment, the electrolyte 28 may further comprise an anti-oxidant. In some embodiments, the anti-oxidant is selected from anti-oxidants that have a lower potential than that of, for example, water. In such embodiments, the selected anti-oxidant is consumed rather than having the hydrolysis of water occur. In some further embodiments, an oxidized form of the anti-oxidant is used at the cathode and a reduced form of the anti-oxidant is used at the anode. Examples of biologically compatible anti-oxidants include, but are not limited to, ascorbic acid (vitamin C), tocopherol (vitamin E), or sodium citrate.

As noted above, the electrolyte 28 may take the form of an aqueous solution housed within a reservoir 26, or in the form of a dispersion in a hydrogel or hydrophilic polymer capable of retaining substantial amount of water. For instance, a suitable electrolyte may take the form of a solution of 0.5 M disodium fumarate: 0.5 M polyacrylic acid: 0.15 M anti-oxidant.

The inner ion selective membrane 30 is generally positioned to separate the electrolyte 28 and the inner active agent reservoir 34, if such a membrane is included within the device. The inner ion selective membrane 30 may take the form of a charge selective membrane. For example, when the active agent 36, 40, 42 comprises a cationic active agent, the inner ion selective membrane 30 may take the form of an anion exchange membrane, selective to substantially pass anions and substantially block cations. The inner ion selective membrane 30 may advantageously prevent transfer of undesirable elements or compounds between the electrolyte 28 and the inner active agent reservoir 34. For example, the inner ion selective membrane 30 may prevent or inhibit the transfer of sodium (Na+) ions from the electrolyte 28, thereby increasing the transfer rate and/or biological compatibility of the iontophoresis device 8.

The inner active agent reservoir 34 is generally positioned between the inner ion selective membrane 30 and the outermost ion selective membrane 38. The inner active agent reservoir 34 may take a variety of forms including any structure capable of temporarily retaining active agent 36. For example, the inner active agent reservoir 34 may take the form of a pouch or other receptacle, a membrane with pores, cavities, or interstices, particularly where the active agent 36 is a liquid. The inner active agent reservoir 34 further may comprise a gel matrix.

Optionally, an outermost ion selective membrane 38 is positioned generally opposed across the active electrode assembly 12 from the active electrode element 24. The outermost membrane 38 may, as in the embodiment illustrated in FIGS. 2A and 2B, take the form of an ion exchange membrane having pores 48 (only one called out in FIGS. 2A and 2B for sake of clarity of illustration) of the ion selective membrane 38 including ion exchange material or groups 50 (only three called out in FIGS. 2A and 2B for sake of clarity of illustration). Under the influence of an electromotive force or current, the ion exchange material or groups 50 selectively substantially passes ions of the same polarity as active agent 36, 40, while substantially blocking ions of the opposite polarity. Thus, the outermost ion exchange membrane 38 is charge selective. Where the active agent 36, 40, 42 is a cation (e.g., lidocaine), the outermost ion selective membrane 38 may take the form of a cation exchange membrane, thus allowing the passage of the cationic active agent while blocking the back flux of the anions present in the biological interface, such as skin.

The outermost ion selective membrane 38 may optionally cache active agent 40. Without being limited by theory, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 38 may take the form of semi-permeable or microporous membrane which is selective by size. In some embodiments, such a semi-permeable membrane may advantageously cache active agent 40, for example by employing the removably releasable outer release liner to retain the active agent 40 until the outer release liner is removed prior to use.

The outermost ion selective membrane 38 may be optionally preloaded with the additional active agent 40, such as ionized or ionizable drugs or therapeutic agents and/or polarized or polarizable drugs or therapeutic agents. Where the outermost ion selective membrane 38 is an ion exchange membrane, a substantial amount of active agent 40 may bond to ion exchange groups 50 in the pores, cavities or interstices 48 of the outermost ion selective membrane 38.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42 may sufficiently cover the outer surface 44 and/or be of sufficient thickness to form a distinct layer 52. In other embodiments, the further active agent 42 may not be sufficient in volume, thickness, or coverage as to constitute a layer in a conventional sense of such term.

The active agent 42 may be deposited in a variety of highly concentrated forms such as, for example, solid form, nearly saturated solution form, or gel form. If in solid form, a source of hydration may be provided, either integrated into the active electrode assembly 12, or applied from the exterior thereof just prior to use.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. Thus, a first type of active agent may be stored in the inner active agent reservoir 34, while a second type of active agent may be cached in the outermost ion selective membrane 38. In such an embodiment, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the active agents 36, 40, 42 will all be of common polarity to prevent the active agents 36, 40, 42 from competing with one another. Other combinations are possible.

The outer release liner may generally be positioned overlying or covering further active agent 42 carried by the outer surface 44 of the outermost ion selective membrane 38. The outer release liner may protect the further active agent 42 and/or outermost ion selective membrane 38 during storage, prior to application of an electromotive force or current. The outer release liner may be a selectively releasable liner made of waterproof material, such as release liners commonly associated with pressure sensitive adhesives.

An interface-coupling medium (not shown) may be employed between the electrode assembly and the biological interface 18. The interface-coupling medium may take, for example, the form of an adhesive and/or gel. The gel may take, for the form of a hydrating gel. Selection of suitable bioadhesive gels is within the knowledge of one skilled in the relevant art.

In the embodiment illustrated in FIGS. 2A and 2B, the counter electrode assembly 14 comprises, from an interior 64 to an exterior 66 of the counter electrode assembly 14: a counter electrode element 68, an electrolyte reservoir 70 storing an electrolyte 72, an inner ion selective membrane 74, an optional buffer reservoir 76 storing buffer material 78, an optional outermost ion selective membrane 80, and an optional outer release liner (not shown).

The counter electrode element 68 is electrically coupled to a second pole 16b of the power source 16, the second pole 16b having an opposite polarity to the first pole 16a. In one embodiment, the counter electrode element 68 is an inert electrode. For example, the counter electrode element 68 may take the form of the carbon-based electrode element discussed above.

The electrolyte reservoir 70 may take a variety of forms including any structure capable of retaining electrolyte 72, and in some embodiments may even be the electrolyte 72 itself, for example, where the electrolyte 72 is in a gel, semi-solid or solid form. For example, the electrolyte reservoir 70 may take the form of a pouch or other receptacle, or a membrane with pores, cavities, or interstices, particularly where the electrolyte 72 is a liquid.

The electrolyte 72 is generally positioned between the counter electrode element 68 and the outermost ion selective membrane 80, proximate the counter electrode element 68. As described above, the electrolyte 72 may provide ions or donate charges to prevent or inhibit the formation of gas bubbles (e.g., hydrogen or oxygen, depending on the polarity of the electrode) on the counter electrode element 68 and may prevent or inhibit the formation of acids or bases or neutralize the same, which may enhance efficiency and/or reduce the potential for irritation of the biological interface 18.

The inner ion selective membrane 74 is positioned between and/or to separate, the electrolyte 72 from the buffer material 78. The inner ion selective membrane 74 may take the form of a charge selective membrane, such as the illustrated ion exchange membrane that substantially allows passage of ions of a first polarity or charge while substantially blocking passage of ions or charge of a second, opposite polarity. The inner ion selective membrane 74 will typically pass ions of opposite polarity or charge to those passed by the outermost ion selective membrane 80 while substantially blocking ions of like polarity or charge. Alternatively, the inner ion selective membrane 74 may take the form of a semi-permeable or microporous membrane that is selective based on size.

The inner ion selective membrane 74 may prevent transfer of undesirable elements or compounds into the buffer material 78. For example, the inner ion selective membrane 74 may prevent or inhibit the transfer of hydroxy (OH—) or chloride (Cl—) ions from the electrolyte 72 into the buffer material 78.

The optional buffer reservoir 76 is generally disposed between the electrolyte reservoir and the outermost ion selective membrane 80. The buffer reservoir 76 may take a variety of forms capable of temporarily retaining the buffer material 78. For example, the buffer reservoir 76 may take the form of a cavity, a porous membrane, or a gel. The buffer material 78 may supply ions for transfer through the outermost ion selective membrane 42 to the biological interface 18. Consequently, the buffer material 78 may comprise, for example, a salt (e.g., NaCl).

The outermost ion selective membrane 80 of the counter electrode assembly 14 may take a variety of forms. For example, the outermost ion selective membrane 80 may take the form of a charge selective ion exchange membrane. Typically, the outermost ion selective membrane 80 of the counter electrode assembly 14 is selective to ions with a charge or polarity opposite to that of the outermost ion selective membrane 38 of the active electrode assembly 12. The outermost ion selective membrane 80 is therefore an anion exchange membrane, which substantially passes anions and blocks cations, thereby prevents the back flux of the cations from the biological interface. Examples of suitable ion exchange membranes include the previously discussed membranes.

Alternatively, the outermost ion selective membrane 80 may take the form of a semi-permeable membrane that substantially passes and/or blocks ions based on size or molecular weight of the ion.

The outer release liner (not shown) may generally be positioned overlying or covering an outer surface 84 of the outermost ion selective membrane 80. The outer release liner may protect the outermost ion selective membrane 80 during storage, prior to application of an electromotive force or current. The outer release liner may be a selectively releasable liner made of waterproof material, such as release liners commonly associated with pressure sensitive adhesives. In some embodiments, the outer release liner may be coextensive with the outer release liner (not shown) of the active electrode assembly 12.

The iontophoresis device 8 may further comprise an inert molding material 86 adjacent exposed sides of the various other structures forming the active and counter electrode assemblies 12, 14. The molding material 86 may advantageously provide environmental protection to the various structures of the active and counter electrode assemblies 12, 14. Enveloping the active and counter electrode assemblies 12, 14 is a housing material 90.

As best seen in FIG. 2B, the active and counter electrode assemblies 12, 14 are positioned on the biological interface 18. Positioning on the biological interface may close the circuit, allowing electromotive force to be applied and/or current to flow from one pole 16a of the power source 16 to the other pole 16b, via the active electrode assembly, biological interface 18 and counter electrode assembly 14.

In use, the outermost active electrode ion selective membrane 38 may be placed directly in contact with the biological interface 18. Alternatively, an interface-coupling medium (not shown) may be employed between the outermost active electrode ion selective membrane 22 and the biological interface 18. The interface-coupling medium may take, for example, the form of an adhesive and/or gel. The gel may take, for example, the form of a hydrating gel or a hydrogel. If used, the interface-coupling medium should be permeable by the active agent 36, 40, 42.

In some embodiments, the power source 16 is selected to provide sufficient voltage, current, and/or duration to ensure delivery of the one or more active agents 36, 40, 42 from the reservoir 34 and across a biological interface (e.g., a membrane) to impart the desired physiological effect. The power source 16 may take the form of one or more chemical battery cells, super- or ultra-capacitors, fuel cells, secondary cells, thin film secondary cells, button cells, lithium ion cells, zinc air cells, nickel metal hydride cells, and the like. The power source 16 may, for example, provide a voltage of 12.8 V DC, with tolerance of 0.8 V DC, and a current of 0.3 mA. The power source 16 may be selectively, electrically coupled to the active and counter electrode assemblies 12, 14 via a control circuit, for example, via carbon fiber ribbons. The iontophoresis device 8 may include discrete and/or integrated circuit elements to control the voltage, current, and/or power delivered to the electrode assemblies 12, 14. For example, the iontophoresis device 8 may include a diode to provide a constant current to the electrode elements 24, 68.

As suggested above, the one or more active agents 36, 40, 42 may take the form of one or more ionic, cationic, ionizeable, and/or neutral drugs or other therapeutic agents. Consequently, the poles or terminals of the power source 16 and the selectivity of the outermost ion selective membranes 38, 80 and inner ion selective membranes 30, 74 are selected accordingly.

During iontophoresis, the electromotive force across the electrode assemblies, as described, leads to a migration of charged active agent molecules, as well as ions and other charged components, through the biological interface into the biological tissue. This migration may lead to an accumulation of active agents, ions, and/or other charged components within the biological tissue beyond the interface. During iontophoresis, in addition to the migration of charged molecules in response to repulsive forces, there is also an electroosmotic flow of solvent (e.g., water) through the electrodes and the biological interface into the tissue. In certain embodiments, the electroosmotic solvent flow enhances migration of both charged and uncharged molecules. Enhanced migration via electroosmotic solvent flow may occur particularly with increasing size of the molecule.

In certain embodiments, the active agent may be a higher molecular weight molecule. In certain aspects, the molecule may be a polar polyelectrolyte. In certain other aspects, the molecule may be lipophilic. In certain embodiments, such molecules may be charged, may have a low net charge, or may be uncharged under the conditions within the active electrode. In certain aspects, such active agents may migrate poorly under the iontophoretic repulsive forces, in contrast to the migration of small more highly charged active agents under the influence of these forces. These higher molecular weight active agents may thus be carried through the biological interface into the underlying tissues primarily via electroosmotic solvent flow. In certain embodiments, the high molecular weight polyelectrolytic active agents may be proteins, polypeptides, or nucleic acids. In other embodiments, the active agent may be mixed with another agent to form a complex capable of being transported across the biological interface via one of the motive methods described above.

In some embodiments, the transdermal drug delivery system 6 includes an iontophoretic drug delivery device 8 for providing transdermal delivery of one or more therapeutic active agents 36, 40, 42 to a biological interface 18. The delivery device 8 includes active electrode assembly 12 including at least one active agent reservoir and at least one active electrode element operable to provide an electromotive force to drive an active agent from the at least one active agent reservoir. The delivery device 8 may include a counter electrode assembly 14 including at least one counter electrode element 68, and a power source 16 electrically coupled to the at least one active and the at least one counter electrode elements 20, 68. In some embodiments, the iontophoretic drug delivery 8 may further include one or more active agents 36, 40, 42 loaded in the at least one active agent reservoir 34.

As shown in FIG. 2C, the delivery device 8 may further include a substrate 10 including a plurality of microneedles 17 in fluidic communication with the active electrode assembly 12, and positioned between the active electrode assembly 12 and the biological interface 18. The substrate 10 may be positioned between the active electrode assembly 12 and the biological interface 18. In some embodiments, the at least one active electrode element 20 is operable to provide an electromotive force to drive an active agent 36, 40, 42 from the at least one active agent reservoir 34, through the plurality of microneedles 17, and to the biological interface 18.

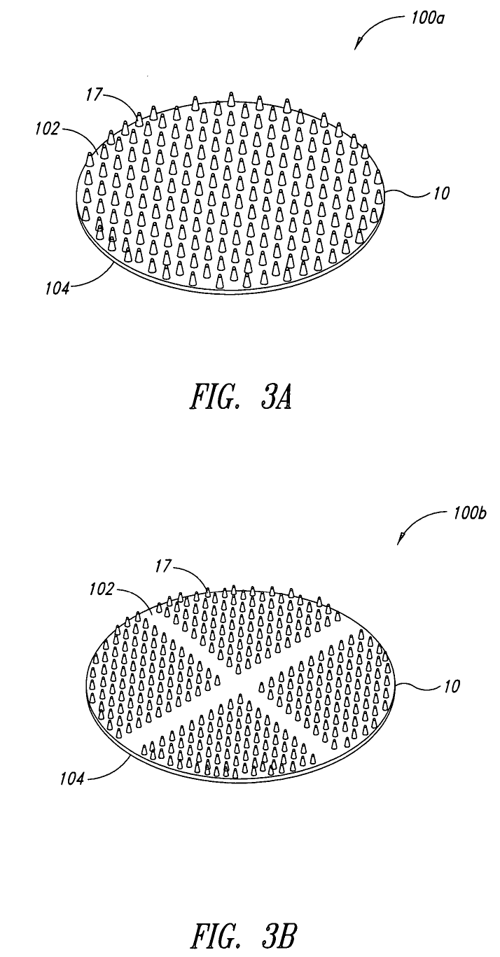

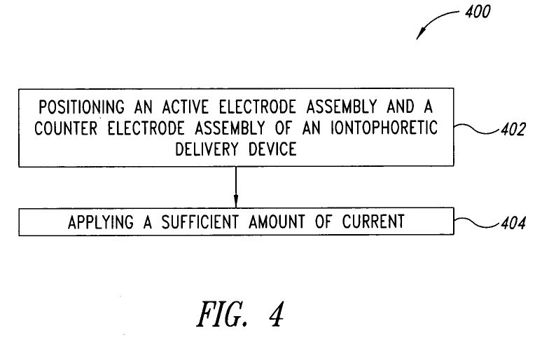

As shown in FIGS. 3A and 3B, the substrate 10 includes a first side 102 and a second side 104 opposing the first side 102. The first side 102 of the substrate 10 includes a plurality of microneedles 17 projecting outwardly from the first side 102. The microneedles 17 may be individually provided or formed as part of one or more arrays. In some embodiments, the microneedles 17 are integrally formed from the substrate 10. The microneedles 17 may take a solid and permeable form, a solid and semi-permeable form, and/or a solid and non-permeable form. In some other embodiments, solid, non-permeable, microneedles may further comprise grooves along their outer surfaces for aiding the transdermal delivery of one or more active agents. In some other embodiments, the microneedles 17 may take the form of hollow microneedles. In some embodiments, the hollow microneedles may be filled with ion exchange material, ion selective materials, permeable materials, semi-permeable materials, solid materials, and the like.

The microneedles 17 are used, for example, to deliver a variety of pharmaceutical compositions, molecules, compounds, active agents, and the like to a living body via a biological interface, such as skin or mucous membrane. In certain embodiments, pharmaceutical compositions, molecules, compounds, active agents, and the like may be delivered into or through the biological interface. For example, in delivering pharmaceutical compositions, molecules, compounds, active agents, and the like via the skin, the length of the microneedle 17, either individually or in arrays 100a, 100b, and/or the depth of insertion may be used to control whether administration of a pharmaceutical compositions, molecules, compounds, active agents, and the like is only into the epidermis, through the epidermis to the dermis, or subcutaneous. In certain embodiments, the microneedle 17 may be useful for delivering high-molecular weight active agents, such as those comprising proteins, peptides and/or nucleic acids, and corresponding compositions thereof. In certain embodiments, for example, wherein the fluid is an ionic solution, the microneedles 17 can provide electrical continuity between the power source 16 and the tips of the microneedles 17. In some embodiments, the microneedles 17, either individually or in arrays 100a, 100b, may be used to dispense, deliver, and/or sample fluids through hollow apertures, through the solid permeable or semi permeable materials, or via external grooves. The microneedles 17 may further be used to dispense, deliver, and/or sample pharmaceutical compositions, molecules, compounds, active agents, and the like by iontophoretic methods, as disclosed herein.

Accordingly, in certain embodiments, for example, a plurality of microneedles 17 in an array 100a, 100b may advantageously be formed on an outermost biological interface-contacting surface of a transdermal drug delivery system 6. In some embodiments, the pharmaceutical compositions, molecules, compounds, active agents, and the like delivered or sampled by such a system 6 may comprise, for example, high-molecular weight active agents, such as proteins, peptides, and/or nucleic acids.

In some embodiments, a plurality of microneedles 17 may take the form of a microneedle array 100a, 100b. The microneedle array 100a, 100b may be arranged in a variety of configurations and patterns including, for example, a rectangle, a square, a circle (as shown in FIG. 3A), a triangle, a polygon, a regular or irregular shapes, and the like. The microneedles 17 and the microneedle arrays 100a, 100b may be manufactured from a variety of materials, including ceramics, elastomers, epoxy photoresist, glass, glass polymers, glass/polymer materials, metals (e.g., chromium, cobalt, gold, molybdenum, nickel, stainless steel, titanium, tungsten steel, and the like), molded plastics, polymers, biodegradable polymers, non-biodegradable polymers, organic polymers, inorganic polymers, silicon, silicon dioxide, polysilicon, silicon rubbers, silicon-based organic polymers, superconducting materials (e.g., superconductor wafers, and the like), and the like, as well as combinations, composites, and/or alloys thereof. Techniques for fabricating the microneedles 17 are well known in the art and include, for example, electro-deposition, electro-deposition onto laser-drilled polymer molds, laser cutting and electro-polishing, laser micromachining, surface micro-machining, soft lithography, x-ray lithography, LIGA techniques (e.g., X-ray lithography, electroplating, and molding), injection molding, conventional silicon-based fabrication methods (e.g., inductively coupled plasma etching, wet etching, isotropic and anisotropic etching, isotropic silicon etching, anisotropic silicon etching, anisotropic GaAs etching, deep reactive ion etching, silicon isotropic etching, silicon bulk micromachining, and the like), complementary-symmetry/metal-oxide semiconductor (CMOS) technology, deep x-ray exposure techniques, and the like. See for example, U.S. Pat. Nos. 6,256,5330-6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; and 6,939,311. Some or all of the teachings therein may be applied to microneedle devices, their manufacture, and their use in iontophoretic applications. In some techniques, the physical characteristics of the microneedles 17 depend on, for example, the anodization conditions (e.g., current density, etching time, HF concentration, temperature, bias settings, and the like) as well as substrate properties (e.g., doping density, doping orientation, and the like).

The microneedles 17 may be sized and shaped to penetrate the outer layers of skin to increase its permeability and transdermal transport of pharmaceutical compositions, molecules, compounds, active agents, and the like. In some embodiments, the microneedles 17 are sized and shaped with an appropriate geometry and sufficient strength to insert into a biological interface (e.g., the skin or mucous membrane on a subject, and the like), and thereby increase a trans-interface (e.g., transdermal) transport of pharmaceutical compositions, molecules, compounds, active agents, and the like.

FIG. 4 shows an exemplary method 400 for transdermal administration of at least one cationic, anionic, or ionizable active agent.

At 402, the method includes positioning an active electrode assembly 12 and a counter electrode assembly 14 of an iontophoretic delivery device 8 on a biological interface 18 of a subject. The active electrode assembly 12 includes an active agent reservoir 34 comprising a hydrogel matrix and at least one cationic, anionic, or ionizable active agent 36, 40, 42 cached in the active agent reservoir 34. The hydrogel matrix may include at least one polymer selected from poly(amidoamines), poly(dimethylsiloxanes), poly(hydroxyethyl methacrylates), poly(N-isopropyl acrylamides), poly[1-vinyl-2-pyrrolidinone-co-(2-hydroxyethyl methacrylate)], poly(acrylamides), poly(acrylic acids), poly(methacrylic acids), poly(ethylene glycols), poly(ethylene glycol monomethacrylate), poly(methacryloyloxyethyl 5-amino salicylate), poly(methacrylic acid)-co-poly(ethylene glycol), poly(vinyl alcohols), and poly(vinyl-pyrrolidones), poly[methacrylic acid-co-polyethylene glycol monomethacrylate-co-methacryloyloxyethyl 5-amino salicylate], poly(2-hydroxyethyl methacrylate-co-methyl methacrylate), poly(acrylamides), poly(aminoproly methacrylamides), poly(N-(3-aminopropyl)methacrylamide), and poly(N,N-dimethy-2-aminoethyl methacrylate), or copolymers, block copolymers, graft copolymers, and heteropolymers thereof, or combinations thereof.

In some embodiments, the one or more polymeric units of the least one polymer are modified with one or more groups selected from charge functional groups, hydrophobic functional groups, hydrophilic functional groups, chemically reactive functional groups, organofunctional group, and bio-compatible groups. In some embodiments, at least a portion of a polymeric backbone of the least one polymer has been modified with one or more groups selected from carboxylate groups, sulfonate groups, amine groups, quaternary amine groups, alkoxy amines, aspartic acids, iminodiacetic acids, and glutamic acids. In some other embodiments, the least one polymer is selected from backbone-modified hydroxyethyl methacrylate polymers, backbone-modified poly(acrylamides), or backbone-modified poly(vinyl alcohol) having one or more backbone units modified with one or more groups selected from carboxylate groups, sulfonate groups, amine groups, quaternary amine groups, alkoxy amines, aspartic acids, iminodiacetic acids, and glutamic acids. In yet some other embodiments, one or more polymeric units of the least one polymer are modified with one or more groups selected from carboxylate groups, sulfonate groups, amine groups, quaternary amine groups, alkoxy amines, aspartic acids, iminodiacetic acids, and glutamic acids.

In some embodiments, the active electrode assembly 12 includes an active agent reservoir 34 comprising at least one analgesic or anesthetic active agent 36, 40, 42 carried by a pharmaceutically acceptable vehicle and included in the hydrogel matrix.

At 404, the method includes applying a sufficient amount of current to transport the at least one cationic, anionic, or ionizable active agent from the active agent reservoir, to the biological interface of the subject, and to administer a therapeutically effective amount of the at least one cationic, anionic, or ionizable active agent.

In some embodiments, the at least one cationic, anionic, or ionizable active agent is selected from immuno-adjuvants, immuno-modulators, immuno-response agents, immuno-stimulators, specific immuno-stimulators, non-specific immuno-stimulators, immuno-suppressants, vaccines, agonists, antagonist, opioid agonist, opioid antagonist, antigens, adjuvants, immunological adjuvants, immunogens, tolerogens, allergens, toll-like receptor agonists, and toll-like receptor antagonists, or combinations thereof.

In some embodiments, applying a sufficient amount of current to transport to transport the at least one cationic, anionic, or ionizable active agent includes providing sufficient voltage and current to deliver a therapeutically effective amount of the at least one cationic, anionic, or ionizable active agent; from the active agent reservoir to the biological interface of the subject. In some other embodiments, applying a sufficient amount of current to transport to transport the at least one cationic, anionic, or ionizable active agent includes providing a sufficient voltage and current to the active electrode assembly 12 to substantially achieve sustained-delivery or controlled-delivery of the at least one cationic, anionic, or ionizable active agent 36, 40, 42 from the active agent reservoir to the biological interface of the subject.

The above description of illustrated embodiments, including what is described in the Abstract, is not intended to be exhaustive or to limit the claims to the precise forms disclosed. Although specific embodiments and examples are described herein for illustrative purposes, various equivalent modifications can be made without departing from the spirit and scope of the disclosure, as will be recognized by those skilled in the relevant art. The teachings provided herein can be applied to other agent delivery systems and devices, not necessarily the exemplary iontophoresis active agent system and devices generally described above. For instance, some embodiments may include additional structure. For example, some embodiments may include a control circuit or subsystem to control a voltage, current, or power applied to the active and counter electrode elements 20, 68. Also for example, some embodiments may include an interface layer interposed between the outermost active electrode ion selective membrane 22 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240,995, herein incorporated in their entirety by reference. Further examples of such hydrogels are disclosed in U.S. patent application Nos. 2004/166147; 2004/105834; and 2004/247655, herein incorporated in their entirety by reference. Product brand names of various hydrogels and hydrogel sheets include Corplex™ by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd.

In certain embodiments, compounds or compositions can be delivered by an iontophoresis device 8 comprising an active electrode assembly 12 and a counter electrode assembly 14, electrically coupled to a power source 16 to deliver an active agent to, into, or through a biological interface 18. The active electrode assembly 12 includes the following: a first electrode member connected to a positive electrode of the power source; an active agent reservoir having a drug solution that is in contact with the first electrode member and to which is applied a voltage via the first electrode member; a biological interface contact member, which may be a microneedle array and is placed against the forward surface of the active agent reservoir; and a first cover or container that accommodates these members. The counter electrode assembly includes the following: a second electrode member connected to a negative electrode of the voltage source; a second electrolyte holding part that holds an electrolyte that is in contact with the second electrode member and to which voltage is applied via the second electrode member; and a second cover or container that accommodates these members.

In certain other embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the voltage source; a first electrolyte reservoir having an electrolyte that is in contact with the first electrode member and to which is applied a voltage via the first electrode member; a first anion-exchange membrane that is placed on the forward surface of the first electrolyte holding part; an active agent reservoir that is placed against the forward surface of the first anion-exchange membrane; a biological interface contacting member, which may be a microneedle array and is placed against the forward surface of the active agent reservoir; and a first cover or container that accommodates these members. The counter electrode assembly includes the following: a second electrode member connected to a negative electrode of the voltage source; a second electrolyte holding part having an electrolyte that is in contact with the second electrode member and to which is applied a voltage via the second electrode member; a cation-exchange membrane that is placed on the forward surface of the second electrolyte reservoir; a third electrolyte reservoir that is placed against the forward surface of the cation-exchange membrane and holds an electrolyte to which a voltage is applied from the second electrode member via the second electrolyte holding part and the cation-exchange membrane; a second anion-exchange membrane placed against the forward surface of the third electrolyte reservoir; and a second cover or container that accommodates these members.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to:

Japanese patent application Serial No. H03-86002, filed Mar. 27, 1991, having Japanese Publication No. H04-297277, issued on Mar. 3, 2000 as Japanese Patent No. 3040517;

Japanese patent application Serial No. 11-033076, filed Feb. 10, 1999, having Japanese Publication No. 2000-229128;

Japanese patent application Serial No. 11-033765, filed Feb. 12, 1999, having Japanese Publication No. 2000-229129;

Japanese patent application Serial No. 11-041415, filed Feb. 19, 1999, having Japanese Publication No. 2000-237326;

Japanese patent application Serial No. 11-041416, filed Feb. 19, 1999, having Japanese Publication No. 2000-237327;

Japanese patent application Serial No. 11-042752, filed Feb. 22, 1999, having Japanese Publication No. 2000-237328;

Japanese patent application Serial No. 11-042753, filed Feb. 22, 1999, having Japanese Publication No. 2000-237329;

Japanese patent application Serial No. 11-099008, filed Apr. 6, 1999, having Japanese Publication No. 2000-288098;

Japanese patent application Serial No. 11-099009, filed Apr. 6, 1999, having Japanese Publication No. 2000-288097;

PCT patent application WO 2002JP4696, filed May 15, 2002, having PCT Publication No WO03037425;

U.S. patent application Ser. No. 10/488,970, filed Mar. 9, 2004;

Japanese patent application 2004/317317, filed Oct. 29, 2004;

U.S. provisional patent application Ser. No. 60/627,952, filed Nov. 16, 2004;

Japanese patent application Serial No. 2004-347814, filed Nov. 30, 2004;

Japanese patent application Serial No. 2004-357313, filed Dec. 9, 2004;

Japanese patent application Serial No. 2005-027748, filed Feb. 3, 2005;

Japanese patent application Serial No. 2005-081220, filed Mar. 22, 2005;

U.S. Provisional Patent Application No. 60/722,789 filed Sep. 30, 2005;

U.S. Provisional Patent Application No. 60/754,688 filed Dec. 29, 2005;

U.S. Provisional Patent Application No. 60/755,199 filed Dec. 30, 2005; and

U.S. Provisional Patent Application No. 60/755,401 filed Dec. 30, 2005.

As one skill in the relevant art would readily appreciate, the present disclosure comprises methods of treating a subject by any of the compositions and/or methods described herein.

Aspects of the various embodiments can be modified, if necessary, to employ systems, circuits and concepts of the various patents, applications and publications to provide yet further embodiments, including those patents and applications identified herein. While some embodiments may include all of the membranes, reservoirs and other structures discussed above, other embodiments may omit some of the membranes, reservoirs, or other structures. Still other embodiments may employ additional ones of the membranes, reservoirs, and structures generally described above. Even further embodiments may omit some of the membranes, reservoirs and structures described above while employing additional ones of the membranes, reservoirs and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

What is claimed is:

1. An iontophoretic drug delivery device for providing transdermal delivery of one or more therapeutic active agents to a biological interface, comprising:
an active electrode assembly including at least one active agent reservoir and at least one active electrode element operable to provide an electromotive force for driving one or more therapeutic agents from the at least one active agent reservoir to the biological interface,
the at least one active agent reservoir comprising a hydrogel matrix having a surface, the hydrogel matrix comprising at least one polymer selected from poly(amidoamines), poly(dimethylsiloxanes), poly(hydroxyethyl methacrylates), poly(N-isopropyl acrylamides), poly[1-vinyl-2-pyrrolidinone-co-(2-hydroxyethyl methacrylate)], poly(acrylamides), poly(acrylic acids), poly(methacrylic acids), poly(ethylene glycols), poly(ethylene glycol monomethacrylate), poly(methacryloyloxyethyl 5-amino salicylate), poly(methacrylic acid)-co-poly(ethylene glycol), poly(vinyl alcohols), and poly(vinyl-pyrrolidones), poly[methacrylic acid-co-polyethylene glycol monomethacrylate-co-methacryloyloxyethyl 5-amino salicylate], poly(2-hydroxyethyl methacrylate-co-methyl methacrylate), poly(acrylamides), poly(aminoproly methacrylamides), poly(N-(3-aminopropyl)methacrylamide), and poly(N,N-dimethy-2-aminoethyl methacrylate), or copolymers, block copolymers, graft copolymers, and heteropolymers thereof, or combinations thereof.

2. The iontophoretic drug delivery device of claim 1 wherein one or more polymeric units of the least one polymer are modified with one or more groups selected from charge functional groups, hydrophobic functional groups, hydrophilic functional groups, chemically reactive functional groups, organofunctional group, and bio-compatible groups.

3. The iontophoretic drug delivery device of claim 1 wherein at least a portion of a polymeric backbone of the least one polymer has been modified with one or more groups selected from carboxylate groups, sulfonate groups, amine groups, quaternary amine groups, alkoxy amines, aspartic acids, iminodiacetic acids, and glutamic acids.

4. The iontophoretic drug delivery device of claim 1 wherein the least one polymer is selected from backbone-modified hydroxyethyl methacrylate polymers, backbone-modified poly(acrylamides), or backbone-modified poly(vinyl alcohol) having one or more backbone units modified with one or more groups selected from carboxylate groups, sulfonate groups, amine groups, quaternary amine groups, alkoxy amines, aspartic acids, iminodiacetic acids, and glutamic acids.

5. The iontophoretic drug delivery device of claim 1, further comprising a therapeutically effective amount of one or more active agents cached in the hydrogel matrix of the at least one active agent reservoir.

6. The iontophoretic drug delivery device of claim 5 wherein the one or more active agents are selected from immuno-adjuvants, immuno-modulators, immuno-response agents, immuno-stimulators, specific immuno-stimulators, non-specific immuno-stimulators, and immuno-suppressants, or combinations thereof.

7. The iontophoretic drug delivery device of claim 5 wherein the one or more active agents are selected from vaccines, agonists, antagonist, opioid agonist, opioid antagonist, antigens, adjuvants, immunological adjuvants, immunogens, tolerogens, allergens, toll-like receptor agonists, and toll-like receptor antagonists, or combinations thereof.

8. The iontophoretic drug delivery device of claim 5 wherein the one or more active agents are selected from analgesics, anesthetics, or combinations thereof.

9. The iontophoretic drug delivery device of claim 5 wherein the one or more therapeutic active agents are selected from cationic, anionic, ionizable, or neutral active agents.

10. The iontophoretic drug delivery device of claim 5 wherein the one or more therapeutic active agents are selected form cationic active agents, and one or more polymeric units of the at least on polymer are modified with negatively charged functional groups.

11. The iontophoretic drug delivery device of claim 5 wherein a substantial portion of the one or more therapeutic active agents are carried by a portion of the surface of the hydrogel matrix, prior to use, in the absence of an electromotive force or current.

12. An iontophoretic drug delivery device for providing transdermal delivery of one or more therapeutic active agents to a biological interface, comprising:
an active electrode assembly including at least one active electrode element, at least one inner active agent reservoir, and an outermost active agent reservoir, the at least one inner active agent reservoir positioned between the at least one active electrode element and the outermost active agent reservoir, the active electrode assembly operable to provide an electrical potential,
the outermost active agent reservoir comprising a hydrogel matrix having a surface, the hydrogel matrix comprising at least one polymer selected from poly(amidoamines), poly(dimethylsiloxanes), poly(hydroxyethyl methacrylates), poly(N-isopropyl acrylamides), poly[1-vinyl-2-pyrrolidinone-co-(2-hydroxyethyl methacrylate)], poly(acrylamides), poly(acrylic acids), poly(methacrylic acids), poly(ethylene glycols), poly(ethylene glycol monomethacrylate), poly(methacryloyloxyethyl 5-amino salicylate), poly(methacrylic acid)-co-poly(ethylene glycol), poly(vinyl alcohols), and poly(vinyl-pyrrolidones), poly[methacrylic acid-co-polyethylene glycol monomethacrylate-co-methacryloyloxyethyl 5-amino salicylate], poly(2-hydroxyethyl methacrylate-co-methyl methacrylate), poly(acrylamides), poly(aminoproly methacrylamides), poly(N-(3-aminopropyl)methacrylamide), and poly(N,N-dimethy-2-aminoethyl methacrylate), or copolymers, block copolymers, graft copolymers, and heteropolymers thereof, or combinations thereof.

13. The iontophoretic drug delivery device of claim 12 wherein one or more polymeric units of the least one polymer are modified with one or more groups selected from carboxylate groups, sulfonate groups, amine groups, quaternary amine groups, alkoxy amines, aspartic acids, iminodiacetic acids, and glutamic acids.

14. The iontophoretic drug delivery device of claim 12, further comprising a therapeutically effective amount of one or more active agents included in the outermost active agent reservoir and the at least one inner active agent reservoir.

15. The iontophoretic drug delivery device of claim 14 wherein a portion of the one or more therapeutic active agents are carried by at least a portion of the surface of the hydrogel matrix, prior to use, and in the absence of an electromotive force or current.

16. The iontophoretic drug delivery device of claim 14 wherein a substantial portion of the one or more therapeutic active agents are cached in the at least one inner active agent reservoir.

17. The iontophoretic drug delivery device of claim 12, further comprising:
a therapeutically effective amount of one or more active agents cached in the at least one inner active agent; the one or more active agents selected from immuno-adjuvants, immuno-modulators, immuno-response agents, immuno-stimulators, specific immuno-stimulators, non-specific immuno-stimulators, immuno-suppressants, vaccines, agonists, antagonist, opioid agonist, opioid antagonist, antigens, adjuvants, immunological adjuvants, immunogens, tolerogens, allergens, toll-like receptor agonists, and toll-like receptor antagonists, or combinations thereof.

18. The iontophoretic drug delivery device of claim 12, further comprising:
a therapeutically effective amount of one or more active agents of a first polarity cached in the at least one inner active agent reservoir and substantially retained therein in the absence of an electromotive force and transferred outwardly from the at least one inner active agent reservoir in the presence of an electromotive force;
wherein one or more polymeric units of the least one polymer are modified with one or more charge fictional groups of a second polarity, the second polarity opposite to the first polarity; and
wherein the hydrogel matrix is substantially passable by ions having the first polarity and substantially unpassable by ions having the second polarity.

19. The iontophoretic drug delivery device of claim 12 wherein the hydrogel matrix takes the form of a porous gel having an outer surface; and wherein a portion of the one or more therapeutic active agents are carried by the outer surface of the hydrogel matrix, prior to use, in the absence of an electromotive force or current.

20. The iontophoretic drug delivery device of claim 12 wherein the at least one inner active agent reservoir comprises a hydrogel matrix, the hydrogel matrix comprising at least one polymer selected from poly(amidoamines), poly(dimethylsiloxanes), poly(hydroxyethyl methacrylates), poly(N-isopropyl acrylamides), poly[1-vinyl-2-pyrrolidinone-co-(2-hydroxyethyl methacrylate)], poly(acrylamides), poly(acrylic acids), poly(methacrylic acids), poly(ethylene glycols), poly(ethylene glycol monomethacrylate), poly(methacryloyloxyethyl 5-amino salicylate), poly(methacrylic acid)-co-poly(ethylene glycol), poly(vinyl alcohols), and poly(vinyl-pyrrolidones), poly[methacrylic acid-co-polyethylene glycol monomethacrylate-co-methacryloyloxyethyl 5-amino salicylate], poly(2-hydroxyethyl methacrylate-co-methyl methacrylate), poly(acrylamides), poly(aminoproly methacrylamides), poly(N-(3-aminopropyl)methacrylamide), and poly(N,N-dimethy-2-aminoethyl methacrylate), or copolymers, block copolymers, graft copolymers, and heteropolymers thereof, or combinations thereof.

21. A method for transdermal administration of at least one cationic, anionic, or ionizable active agent, comprising:
positioning an active electrode assembly and a counter electrode assembly of an iontophoretic delivery device on a biological interface of a subject, the active electrode assembly including an active agent reservoir comprising a hydrogel matrix and at least one cationic, anionic, or ionizable active agent cached in the active agent reservoir; the hydrogel matrix comprising at least one polymer selected from poly(amidoamines), poly(dimethylsiloxanes), poly(hydroxyethyl methacrylates), poly(N-isopropyl acrylamides), poly[1-vinyl-2-pyrrolidinone-co-(2-hydroxyethyl methacrylate)], poly(acrylamides), poly(acrylic acids), poly(methacrylic acids), poly(ethylene glycols), poly(ethylene glycol monomethacrylate), poly(methacryloyloxyethyl 5-amino salicylate), poly(methacrylic acid)-co-poly(ethylene glycol), poly(vinyl alcohols), and poly(vinyl-pyrrolidones), poly[methacrylic acid-co-polyethylene glycol monomethacrylate-co-methacryloyloxyethyl 5-amino salicylate], poly(2-hydroxyethyl methacrylate-co-methyl methacrylate), poly(acrylamides), poly(aminoproly methacrylamides), poly(N-(3-aminopropyl)methacrylamide), and poly(N,N-dimethy-2-aminoethyl methacrylate), or copolymers, block copolymers, graft copolymers, and heteropolymers thereof, or combinations thereof; and
applying a sufficient amount of current to transport the at least one cationic, anionic, or ionizable active agent from the active agent reservoir, to the biological interface of the subject, and to administer a therapeutically effective amount of the at least one cationic, anionic, or ionizable active agent.

22. The method of claim 21 wherein at least a portion of a polymeric backbone of the least one polymer has been modified with one or more groups selected from carboxylate groups, sulfonate groups, amine groups, quaternary amine groups, alkoxy amines, aspartic acids, iminodiacetic acids, and glutamic acids.

23. The method of claim 21 wherein the at least one cationic, anionic, or ionizable active agent is selected from immuno-adjuvants, immuno-modulators, immuno-response agents, immuno-stimulators, specific immuno-stimulators, non-specific immuno-stimulators, immuno-suppressants, vaccines, agonists, antagonist, opioid agonist, opioid antagonist, antigens, adjuvants, immunological adjuvants, immunogens, tolerogens, allergens, toll-like receptor agonists, and toll-like receptor antagonists, or combinations thereof.

24. The method of claim 21 wherein applying a sufficient amount of current to transport to transport the at least one cationic, anionic, or ionizable active agent includes providing sufficient voltage and current to deliver a therapeutically effective amount of the at least one cationic, anionic, or ionizable active agent; from the active agent reservoir to the biological interface of the subject.

25. The method of claim 21 wherein applying a sufficient amount of current to transport to transport the at least one cationic, anionic, or ionizable active agent includes providing a sufficient voltage and current to the active electrode assembly to substantially achieve sustained-delivery or controlled-delivery of the at least one cationic, anionic, or ionizable active agent from the active agent reservoir to the biological interface of the subject.
